(19) United States
(12) Patent Application Publication
(10) Pub. No.: US 2002/0115605 A1
(43) Pub. Date: Aug. 22, 2002

(54) Novel peptide with effects on cerebral health

(76) Inventors: Matthew During, Philadelphia, PA (US); Colin N. Haile, Philadelphia, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/939,472

(22) Filed: Aug. 24, 2001

Related U.S. Application Data
(60) Provisional application No. 60/227,631, filed on Aug. 24, 2000.

(51) Int. Cl. A61K 38/08

(52) U.S. Cl. 514/12; 514/16; 530/328

(57) ABSTRACT

The present invention involves peptides with memory enhancing activity that are homologous to glucagon, Exendin- and glucagon-like peptides; functional analogs, derivatives, fragments and mimetics of these peptides; methods of synthesizing and modifying such peptides; methods of using such peptides to treat nervous system or neurological disorders and to facilitate learning and memory in mammals; and methods of delivering such peptides to mammals for treatment of nervous system or neurological disorders and for facilitation of learning and memory.

### CONTINUING APPLICATION DATA

This application claim priority under 35 U.S.C. §119 based upon U.S. Provisional Application No. 60/227,631 filed Aug. 24, 2000.

### FIELD OF THE INVENTION

The present invention relates to the field of neurology and to peptides with cognitive enhancing activity and, more particularly, to novel peptides, their functional analogs, derivatives, fragments, and/or their functional mimetics; to methods of synthesizing such peptides; to methods of using such peptides to treat nervous system or neurological disorders and to facilitate learning and memory in mammals; and to methods of administering such peptides to mammals for treatment of nervous system or neurological disorders and for facilitation of learning and memory.

### BACKGROUND OF THE INVENTION

Learning and memory in animals, both vertebrates and invertebrates, involves what is commonly termed as synaptic plasticity, i.e., a mechanism by which a given input is associated with enhanced or facilitated output. The most commonly established physiological model of such learning is long term potentiation (LTP), by which repeated excitatory pulses, i.e., tetanic stimuli, lead to a long lasting potentiation of the stimulated synapse.

The molecular mechanism of this synaptic potentiation and plasticity is starting to be unraveled, with the data suggesting a change in gene expression mediated via transcriptional activation. The transcription factors with the most convincing and supportive data are members of the cAMP responsive element binding protein (CREB) family. Loss of plasticity and impaired learning and memory have been demonstrated in studies involving the delivery of mutant CREB in model systems as well as studies of CREB knockout mice. Conversely, activating CREB or overexpressing CREB has been shown to induce a super-learning phenotype.

The mechanism of CREB activation is via cAMP signaling; hence, there has been a search for drugs and other compounds that facilitate the accumulation of intracellular cAMP. The most commonly identified drugs that show facilitation of cAMP accumulation are phosphodiesterase (PDE) inhibitors. One example, Rolipram, a PDE IV inhibitor, has shown remarkable effects in both facilitating LTP and improving learning and memory.

There are a large number of endogenous peptides that have effects on learning and memory in mammalian model systems. These include vasoactive intestinal protein (VIP), vasopressin or anti-diuretic hormone (ADH), and corticotrophin releasing hormone (CRH). Each of these native peptides, however, retains pleiotropic actions, including influences on neuroendocrine function, as well as potential anxiogenic or arousal effects that are likely to limit any potential applications. Moreover, these peptides generally are only effective if directly delivered into the central nervous system (CNS).

One family of peptides that does not appear to be associated with central effects on the brain and nervous system yet whose members activate cAMP in the periphery are the glucagon-like peptides (GLP). A BLAST (a homology search engine) analysis of GLP and GLP family members was undertaken to pull out the homologous domain of these proteins to determine the possibility of isolating a small (<10 amino acid) peptide that would retain cAMP activation ability, would be more stable, and, most significantly, would pass the blood-brain barrier (BBB). Such a peptide would have cognitive-enhancing efficacy following peripheral administration.

In the instant invention, small peptides were synthesized with the goal of inducing cAMP production for cognitive-enhancing efficacy. The synthetic peptides of the instant invention, their functional analogs, derivatives, fragments, and/or their functional mimetics, have cognitive and learning enchancing activity. These peptides, their functional anlogs, derivatives, fragments, and/or their functional mimetics, can be used to treat nervous system or neurological disorders associated with neuronal loss or dysfunction, including, but not limited to, Parkinson's Disease, Alzheimer's Disease, Huntington's Disease, ALS, stroke, attention deficit disorder (ADD) and neuropsychiatric syndromes, and to facilitate learning, memory, and cognition in mammals. One peptide of the present invention is a peptide with the sequence HSEGTFTSD (SEQ. ID. NO:1), hereinafter referred to as Gilatide.

### DEFINITIONS

In the present invention, the terms “functional” or “active” “analogs,” “derivatives,” or “fragments” are used interchangeably to mean a chemical substance that is related structurally and functionally to another substance. An analog, derivative, or fragment contains a modified structure from the parent substance, in this case Gilatide, and maintains the function of the parent substance, in this instance, the biological function or activity of Gilatide in cellular and animal models. The biological activity of the analog, derivative, or fragment may include an improved desired activity or a decreased undesirable activity. The analog, dervative, or fragment need not, but can be synthesized from the other substance. For example, a Gilatide analog means a compound structurally related to Gilatide, but not necessarily made from Gilatide. Analogs, derivatives, or fragments of the instant invention, include, but are not limited to, analogs of the synthetic peptide, Gilatide, that are homologous to glucagon, Exendin- and glucagon-like peptides.

As used herein, the term “peptide,” is used in reference to a functional or active analog, derivative or fragment of Gilatide or a Gilatide-derived peptide, means a compound containing naturally occurring amino acids, non-naturally occurring amino acids or chemically modified amino acids, provided that the compound retains the bioactivity or function of Gilatide.

In the present invention, the terms “functional” or “active” “mimetic” means a Gilatide-derived peptide having a non-amino acid chemical structure that mimics the structure of Gilatide or a Gilatide-derived peptide and retains the bioactivity and function of Gilatide in cellular and animal models. The biological activity or function may include an improved desired activity or a decreased undesirable activity. Such a mimetic generally is characterized as exhibiting similar physical characteristics such as size, charge or hydrophobicity in the same spatial arrangement found in Gilatide or the Gilatide-derived peptide counterpart. A specific example of a peptide mimetic is a compound in which the amide bond between one or more of the amino acids is replaced, for example, by a carbon-carbon bond or other bond well known in the art (see, for example, Sawyer, Peptide Based Drug Design, ACS, Washington (1995), which is incorporated herein by reference).

As used herein, the term “amino acid” refers to one of the twenty naturally occurring amino acids, including, unless stated otherwise, L-amino acids and D-amino acids. The term amino acid also refers to compounds such as chemically modified amino acids including amino acid analogs, naturally occurring amino acids that are not usually incorporated into peptides such as norleucine, and chemically synthesized compounds having properties known in the art to be characteristic of an amino acid, provided that the compound can be substituted within a peptide such that it retains its biological activity. For example, glutamine can be an amino acid analog of asparagine, provided that it can be substituted within an active fragment, derivative or analog of Gilatide that retains its bioactivity or function in cellular and animal models. Other examples of amino acids and amino acids analogs are listed in Gross and Meienhofer, The Peptides: Analysis, Synthesis, Biology, Academic Press, Inc., New York (1983), which is incorporated herein by reference. An amino acid also can be an amino acid mimetic, which is a structure that exhibits substantially the same spatial arrangement of functional groups as an amino acid but does not necessarily have both the α-amino and .α-carboxyl groups characteristic of an amino acid.

“Prophylactic” as used herein means the protection, in whole or in part, against nervous system or neurological diseases, disorders, and conditions associated with neuronal loss or dysfunction.

“Therapeutic” as used herein means the amelioration of, and the protection, in whole or in part, against further, nervous system or neurological diseases, disorders, and conditions associated with neuronal loss or dysfunction.

### ABBREVIATIONS

“LTP” means “long term potentiation”

“GLP” means “glucagon-like protein”

“CREB” means “cAMP responsive element binding protein”

“CNS” means “central nervous system”

“BBB” means “blood-brain barrier”

“PDE” means “phosphodiesterase”

“PAR” means “passive avoidance response”

“VEH” means “vehicle”

“IN” means “intranasal”

“ADD” means “attention deficit disorder”

### BRIEF DESCRIPTION OF THE DRAWINGS

FIG. 1. A bar graph of latency for control rats and rats pretreated with various levels of Gilatide or Vehicle (VEH), where latency is measured in a passive avoidance apparatus. The bar graph shows mean (±S.E.M.) latencies (acquisition) to move into the dark compartment from a bright compartment of a passive avoidance apparatus. The statistically significant data on the group of rats treated with 10 μg versus rats treated with VEH are shown at 1 day, 3 days, 7 days, and 21 days following the aversive stimulus.

FIG. 2. A bar graph of latency for control rats and rats pretreated via various routes of administration of Gilatide or Vehicle (VEH), where latency is measured in a passive avoidance apparatus for a passive avoidance response (PAR). The bar graph shows mean (±S.E.M.) latencies (acquisition) to move into the dark compartment from a bright compartment of a passive avoidance apparatus. ⁺P=0.1; *P=<0.05, (t-test) vs. VEH.

FIG. 3. A bar graph of latency for control rats and rats pretreated with various levels of Gilatide, Vehicle (VEH), or Nicotine, where latency is measured in a passive avoidance apparatus. The bar graph shows mean (±S.E.M.) latencies (retention) to move into the dark compartment from a bright compartment of a passive avoidance apparatus. ⁺P=0.1; *P=<0.05, (t-test) vs. VEH, **P=<0.05 vs. Nicotine.

FIG. 4. A bar graph showing the effects of Gilatide on consolidation of learning for rats pretreated with either Gilatide or Vehicle (VEH), where latency is measured in a passive avoidance apparatus. The bar graph illustrates mean (±S.E.M.) latencies (consolidation) to move into the dark compartment from a bright compartment of a passive avoidance apparatus.

FIG. 5. A bar graph of latency for control rats and rats pretreated with various levels of Gilatide with or without an Exendin-4 antagonist, or vehicle (VEH), where latency is measured in a passive avoidance apparatus. The bar graph illustrates mean (±S.E.M.) latencies to move into the dark compartment from a bright compartment of a passive avoidance apparatus. Co-treatment with the Exendin-4 antagonist (9-39) (10 μg) completely blocked enhancement of associative learning by Gilatide (10 μg) (*P=0.03 vs. Gilatide 10 μg, combination vs. VEH, ##P=0.43). Increasing the dose of Gilatide (20 μg) surmounted the antagonism (vs. VEH, **P=0.04).

FIG. 6. A bar graph of latency for control rats and rats pretreated with Gilatide, saline, scrambled peptide, or vehicle (VEH), where latency is measured in a passive avoidance apparatus. The graph shows mean (±S.E.M.) latencies to move into the dark compartment from a bright compartment of a passive avoidance apparatus.

FIG. 7. A graph showing the effects of Gilatide on locomotor activity of rats. The graph illustrates mean (±S.E.M.) distance traveled (cm) over 30 minutes in rats administered VEH (5% β cyclodextrin) or Gilatide (10-60 μg, intranasal, in 5% β cyclodextrin). Distance traveled did not differ between treatments (P>0.05).

FIG. 8. A bar graph illustrating the effects of Gilatide on nociception based upon the results of a tail immersion assay. The graph shows mean (±S.E.M.) tail flick latencies following pretreatment with VEH (5% β cyclodextrin) or Gilatide (10 μg; intranasal in 5% β cyclodextrin). Latency measures did not differ between treatments (P>0.05).

FIG. 9. A bar graph illustrating the effects of acute administration of Gilatide on food or water intake. The graphs show mean (±S.E.M.) food (A) and water (B) intake in rats following 18 hours of deprivation.

FIG. 10. Graphs illustrating the effects of Gilatide on retention of spatial learning based upon the results of a Morris Water Maze task assay. The graphs show mean (±S.E.M.) latency to find a submerged platform in the Morris Water Maze paradigm. There was no difference in acquisition between groups during training (A). Retention tests (B) 48 hours following training yielded a trend for significance at the 10 μg dose (t=1.774(27); P=0.08) and significant difference between Gilatide 30 μg dose (t=2.76(26); P+0.01) compared to VEH.

FIG. 11. Effects of Gilatide (10 μg, IN) on CREB (A, B) and MAPK (C) immunoreactivity in the hippocampus. Rats were administered either vehicle (V), a dopamine agonist (A), or Gilatide (G).

### DETAILED DESCRIPTION

The instant invention provides evidence that a peptide, Gilatide, has remarkable cognitive-enhancing activity. The peptide is nine amino acids long and has the following amino acid sequence: HSEGTFTSD (SEQ. ID. NO: 1). Gilatide is homologous, but not identical, to fragments of both GLP-1 (amino acids 7-15) as well as Exendin-4 (amino acids 7-15), a peptide isolated from the saliva of the Gila Monster. Where these native proteins have a glycine in position 2, however, the synthetic peptide of the instant invention has a serine in this position. The substitution of serine for glycine in position 2 increases the stability of the synthetic peptide in comparison to that of both GLP-1 and Exendin-4. Of interest, the glucagon protein sequence of both the torpedo and the common dogfish has a serine in the position 2.

The present invention aims at providing Gilatide and analogs, derivatives, fragments, and mimetics thereof as novel pharmaceutical agents for the therapeutic and prophylactic treatment of neurological and nervous system disorders associated with neuronal loss or dysfunction, including, but not limited to, Parkinson's Disease, Alzheimer's Disease, Huntington's Disease, ALS, stroke, ADD, and neuropsychiatric syndromes, and to facilitate learning and cognition in mammals.

Peptides, Analogs, Derivatives and Mimetics Thereof

The instant invention relates to Gilatide and to variations of the Gilatide peptide that show the biological activity or function of Gilatide. This biological activity or function may include an improved activity or a decreased undesirable activity. Such variants of Gilatide include functional analogs, derivatives, fragments, and mimetics of Gilatide. The invention further includes methods for selecting functional analogs, fragments, and mimetics of Gilatide from a collection of randomly obtained or rationally designed candidate compounds. Compounds selected by the process described herein will retain the biological activity or function of Gilatide. Nucleic acids encoding Gilatide and fragments, analogs, derivatives, and mimetics thereof are also provided.

The fragments, derivatives, analogs, or mimetics of the Gilatide peptide may be: (1) one in which one or more of the amino acid residues are substituted with a conserved or non-conserved amino acid residue; (2) one in which one or more of the amino acid residues includes a substituent group; (3) one in which the mature peptide is fused with another compound, such as a compound to increase the half-life of the peptide (for example, polyethylene glycol); (4) one in which the additional amino acids are fused to the mature peptide, such as a leader or secretory sequence or a sequence that is employed for purification of the mature peptide or a propeptide sequence; or (5) one which comprises fewer or greater amino acid residues than has SEQ. ID. NO:1 and yet still retains acitivity characteristics of Gilatide. Such fragments, derivatives, analogs, and mimetics are deemed to be within the scope of those skilled in the art from the teachings herein.

Preparation of Peptides, Analogs, Derivatives and Mimetics Thereof

One skilled in the art may prepare such fragments, derivatives, analogs, or mimetics of the Gilatide peptide by modifying the native sequence by resultant single or multiple amino acid substitutions, additions, or deletions. These changes are preferably of a minor nature, such as conservative amino acid substitutions, that do not significantly affect the folding or activity of the peptide. For instance, one polar amino acid, such as threonine, may be substituted for another polar amino acid, such as serine; or one acidic amino acid, such as aspartic acid, may be substituted for another acidic amino acid, such as glutamic acid; or a basic amino acid, such as lysine, arginine, or histidien, may be substituted for another basic amino acid; or a non-polar amino acid, such as alanine, leucine or isoleucine, may be substituted for another non-polar amino acid. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990). Of course, the number of amino acid substitutions a skilled artisan would make depends on many factors. Moreover, amino acids in the Gilatide peptide of the present invention that are essential for function can be identified by methods known in the art, such as site-directed mutagenesis or alanine-scanning mutagenesis. (Cunningham & Wells, Science 244:1081-1085 (1989)). The latter procedure introduces single alanine mutations at every residue in the molecule. The resultant mutant molecules are then tested for biological activity.

Peptides of the present invention can be prepared in any suitable manner. Such peptides include isolated naturally occurring peptides, recombinantly produced peptides, synthetically produced peptides, or peptides produced by a combination of these methods. Means for preparing such peptides are well known in the art.

Identification of Active Peptides Analogs, Derivatives and Mimetics Thereof

Peptides of the instant invention can be identifed by screening a large collection, or library, of random peptides or peptides of interest. Peptide libraries include, for example, tagged chemical libraries comprising peptides and peptidomimetic molecules. Peptide libraries also comprise those generated by phage display technology. Phage display technology includes the expression of peptide molecules on the surface of phage as well as other methodologies by which a protein ligand is or can be associated with the nucleic acid that encodes it. Methods for the production of phage display libraries, including vectors and methods of diversifying the population of peptides that are expressed, are well known in the art (see, for example, Smith & Scott, Methods Enzymol. 217:228-257 (1993); Scott & Smith, Science 249:386-390 (1990); and Huse, WO 91/07141 and WO 91/07149, each of which is incorporated herein by reference). These or other well known methods can be used to produce a phage display library, from which the displayed peptides can be cleaved and assayed for activity, for example, using the methods disclosed infra. If desired, a population of peptides can be assayed for activity, and an active population can be subdivided and the assay repeated in order to isolate an active peptide from the population. Other methods for producing peptides useful in the invention include, for example, rational design and mutagenesis based on the amino acid sequences of active fragments of Gilatide.

An active analog, derivative, fragment or mimetic of Gilatide useful in the invention can be isolated or synthesized using methods well known in the art. Such methods include recombinant DNA methods and chemical synthesis methods for production of a peptide. Recombinant methods of producing a peptide through expression of a nucleic acid sequence encoding the peptide in a suitable host cell are well known in the art and are described, for example, in Sambrook et al., Molecular Cloning: A Laboratory Manual, 2nd Ed, Vols 1 to 3, Cold Spring Harbor Laboratory Press, New York (1989), which is incorporated herein by reference.

An active analog, derivative, fragment or mimetic of Gilatide useful in the invention also can be produced by chemical synthesis, for example, by the solid phase peptide synthesis method of Merrifield et al., J. Am. Chem. Soc. 85:2149 (1964), which is incorporated herein by reference. Standard solution methods well known in the art also can be used to synthesize a peptide useful in the invention (see, for example, Bodanszky, Principles of Peptide Synthesis, Springer-Verlag, Berlin (1984) and Bodanszky, Peptide Chemistry, Springer-Verlag, Berlin (1993), each of which is incorporated herein by reference). A newly synthesized peptide can be purified, for example, by high performance liquid chromatography (HPLC), and can be characterized using, for example, mass spectrometry or amino acid sequence analysis.

In addition, active analogs, derivatives, fragments or mimetics of Gilatide can be synthesized by use of a peptide synthesizer. Furthermore, if desired, non-classical amino acids or chemical amino acid analogs can be introduced as a substitution or addition into the Gilatide sequence. Non-classical amino acids include but are not limited to the D-isomers of the common amino acids, α-amino isobutyric acid, 4 amino-butyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

Modifications

It is understood that limited modifications can be made to an active analog, derivative, fragment or mimetic of Gilatide without destroying its biological function. Thus, a modification of a functional analog, derivative, fragment or mimetic of Gilatide that does not destroy its activity or function is within the definition of a functional analog, derivative, fragment or mimetic of Gilatide. A modification can include, for example, an addition, deletion, or substitution of amino acid residues; a substitution of a compound that mimics amino acid structure or function; and addition of chemical moieties such as amino or acetyl groups.

A particularly useful modification is one that confers, for example, increased stability. For example, incorporation of one or more D-amino acids or substitution or deletion of lysine can increase the stability of an active analog, derivative, fragment or mimetic of Gilatide by protecting against peptide degradation. The substitution or deletion of a lysine residue confers increased resistance to trypsin-like proteases, as is well known in the art (Partridge, Peptide Drug Delivery to the Brain, Raven Press, New York, 1991). These substitutions increase stability and, thus, bioavailability of peptides, but do not affect activity.

A useful modification also can be one that promotes peptide passage across the blood-brain barrier, such as a modification that increases lipophilicity or decreases hydrogen bonding. For example, a tyrosine residue added to the C-terminus of a peptide may increase hydrophobicity and permeability to the blood-brain barrier (see, for example, Banks et al., Peptides 13:1289-1294 (1992), which is incorporated herein by reference, and Pardridge, supra, 1991). A chimeric peptide-pharmaceutical that has increased biological stability or increased permeability to the blood-brain barrier, for example, also can be useful in the method of the invention.

One skilled in the art can readily assay the ability of an active analog, derivative, fragment or mimetic of Gilatide to cross the blood-brain barrier in vivo, for example using a model of the blood-brain barrier based on a brain microvessel endothelial cell culture system, for example as described in Bowman et al., Ann. Neurol. 14:396-402 (1983) or Takahura et al., Adv. Pharmacol. 22:137-165 (1992), each of which is incorporated herein by reference.

Included within the scope of the invention are active analogs, derivatives, fragments or mimetics of Gilatide that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Moreover, the peptide of the present invention can be a chimeric, or fusion, protein comprising Gilatide or an analog, derivative, fragment, or mimetic thereof joined at its amino- or carboxy-terminus via a peptide bond to an amino acid sequence of a different protein. In one embodiment, such a chimeric protein is produced by recombinant expression of a nucleic acid encoding the protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

Methods and Results

Passive Avoidance Response

In the instant invention, rats were pretreated intranasally with one of three dose levels (10 μg/kg, 30 μg/kg, or 60 μg/kg) of Gilatide in 5% β cyclodextrin or an octamer having a sequence homology to CRH and urocortin. The native forms of these latter peptides previously have been shown to have some potential efficacy in memory facilitation. A control group received vehicle (5% cyclodextrin) alone. With three dose levels for each of the peptides studied, a total of seven (7) groups were employed, each group having 5-8 rats, for a total of 50 rats tested. On the first day of conditioning, the pretreated rats (N=7-13) were administered a single foot shock trial (0.1 mA over 3 seconds) after entering the dark compartment. The animals were replaced in the test apparatus and latencies again were measured on Days 1, 3, 7, and 21 following the aversive stimulus.

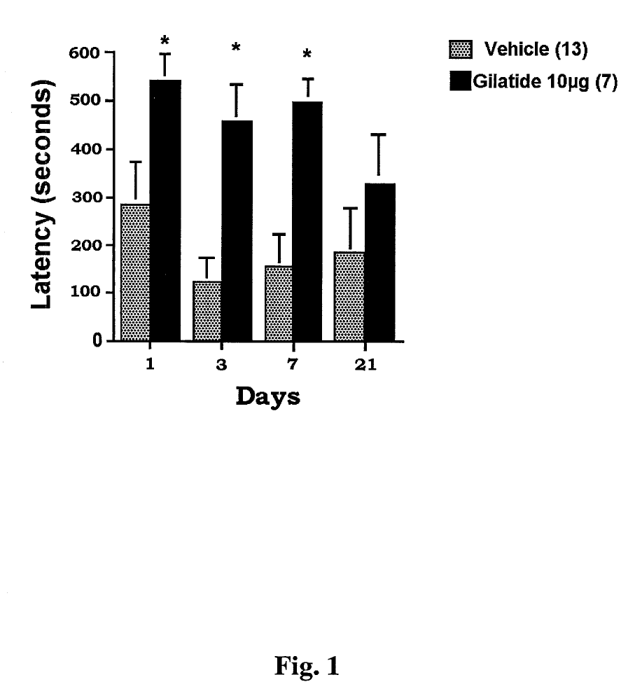

As predicted, the control animals (N=13) showed short latencies to enter the dark room (mean±SEM=15.4±3.8) prior to exposure to the single mild shock. Similarly, all other groups had increased latencies ranging from 14.8 to 31.6 seconds. At 24 hours (Day 1) following the initial test, and delivery of the single shock, the animals were replaced in the test apparatus and latency again measured. Those control rats, which had learned that the aversive stimulation was associated with entering the dark room, had mean latencies of 286.3±88.8 seconds. (FIG. 1) Similarly, all other groups had increased latencies, ranging from 342.5 to 542.9 seconds. Those rats (N=7) that received 10 μg of Gilatide had a mean latency of 542.9 seconds, an increase in latency of 90% above those rats administered vehicle alone. This difference was statistically significant (p<0.05).

On Day 3, rats were again tested in the apparatus. By this time the control rats had started to forget the aversive stimulus; thus, their latencies decreased to 125.6±51.4 seconds. (FIG. 1) Similarly, all other groups, except one, had a drop in latencies, with values ranging from 118.4 to 279 seconds. Of interest, the rats administered 10 μg Gilatide maintained a mean latency of 458 seconds. This result was statistically significant at p=0.003 compared to the rats administered vehicle only. (FIG. 1)

On Day 7 following delivery of the peptide, the rats were again placed in the test apparatus. The rats administered 10 μg Gilatide had a mean latency of 501.1 seconds compared to the control (vehicle only) group, which had a mean latency of 157.6 (p=0.002). (FIG. 1)

Finally, the effect was tested 21 days after the single episode of training. By this time, the memory facilitation was lost, although a trend was apparent even at this markedly delayed time point. (FIG. 1)

Route of Administration Comparison

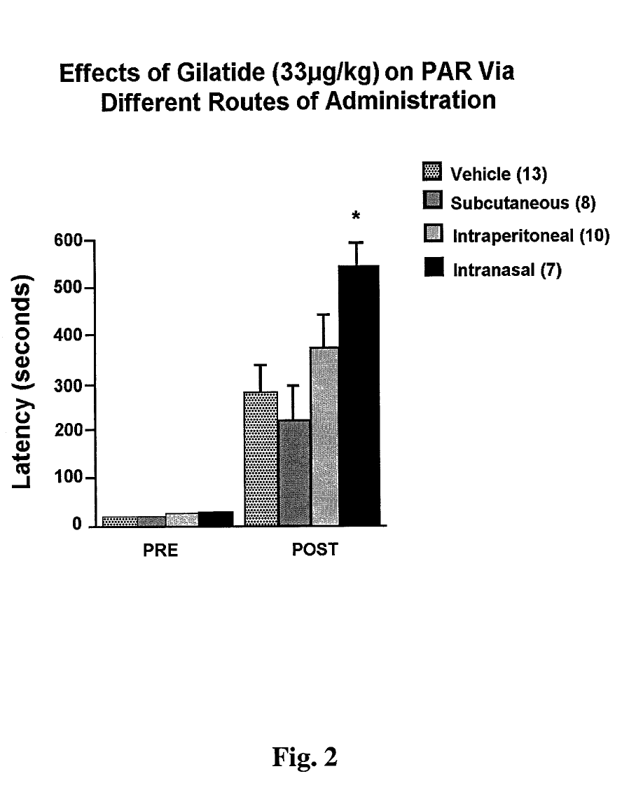

In a second series of experiments, rats were pretreated with either 33 μg/kg Gilatide in 5% β cyclodextrin or vehicle by one of three routes of administration: intranasally, subcutaneously, or intraperitoneally. On Day 0, the rats (N=7-13) were conditioned by administration of a single foot shock trial (0.1 mA over 3 seconds) after entry into the dark compartment of a passive avoidance apparatus (the same passive avoidance chamber used in the first series of experiments). At 24 hours (Day 1) following the initial test, and delivery of the single shock, the animals were replaced in the test apparatus and latency again measured. (FIG. 2)

Dose Level

Since the lowest dose of Gilatide tested, 10 μg, was effective, smaller doses were tested to determine the activity of smaller doses in this animal model. Rats (N=5-10) were pretreated intranasally with one of five dose levels (0.1 μg/kg, 1 μg/kg, 3 μg/kg, 30 μg/kg, or 60 μg/kg) of Gilatide in 5% β cyclodextrin, vehicle (5% cyclodextrin), or Nicotine (0.3 mg/kg, subcutaneously). On Day 0, the rats were conditioned by administration of a single foot shock trial (0.1 mA over 3 seconds) after entry into the dark compartment of a passive avoidance apparatus (the same passive avoidance chamber used in the other experiments). The preconditioned rats were retested on Days 1, 3, 7, and 21.

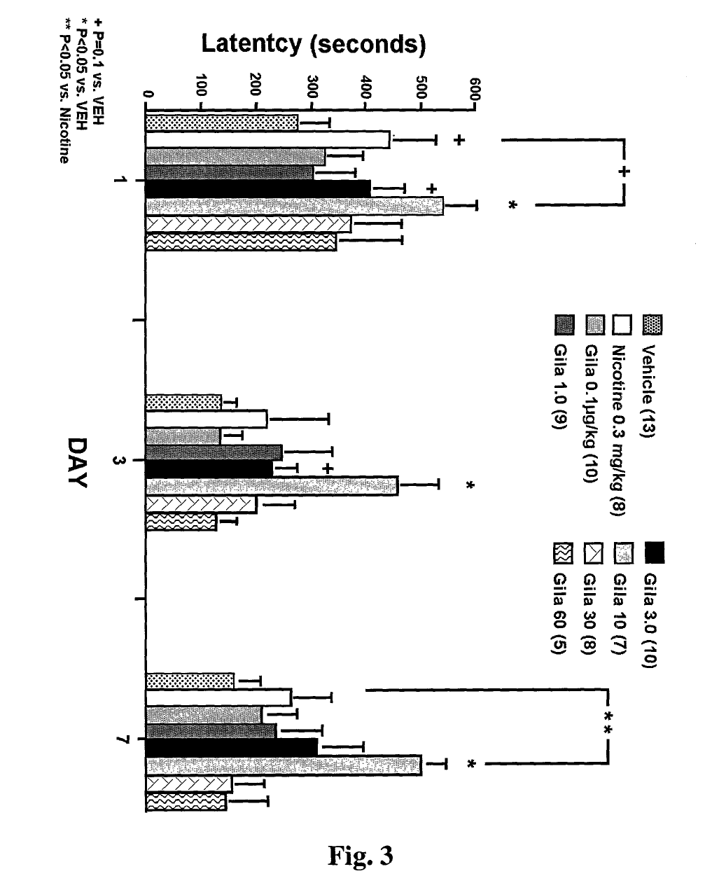

Although the rats administered either 0.1 or 1.0 μg/kg showed no effect, the rats receiving 3.0 μg/kg of Gilatide exhibited extended latencies at 3 and 7 days post conditioning. (FIG. 3) This trend was observed, but the effect did not reach statistical significance. The positive control group (0.3 mg/kg nicotine; the gold standard for this assay and a well-established nicotine dose in this task) exhibited modestly increased latencies at 24 hours. (FIG. 3) This effect, however, was transient and not as significant as the effect of Gilatide administered at 10 μg/kg. The effect was further tested at 21 days post the single episode training. By this time, however, the memory facilitation was lost, although there was a trend even at this markedly delayed time point.

Memory Consolidation

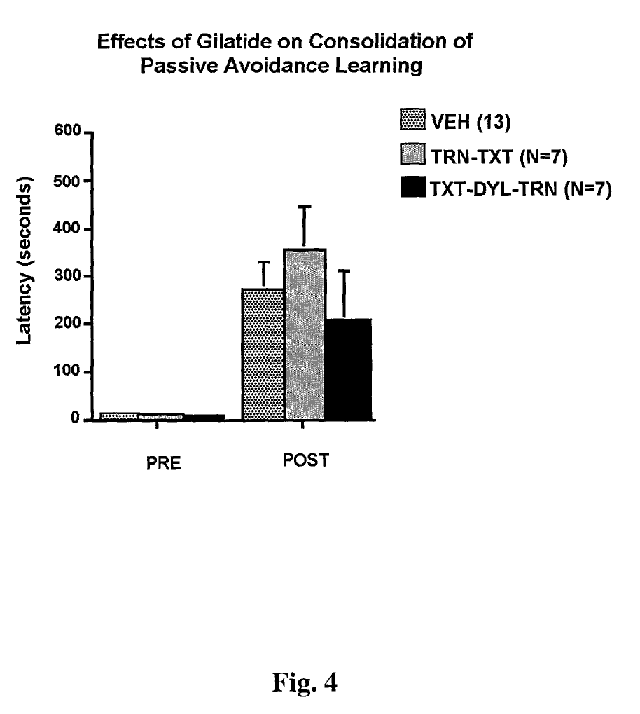

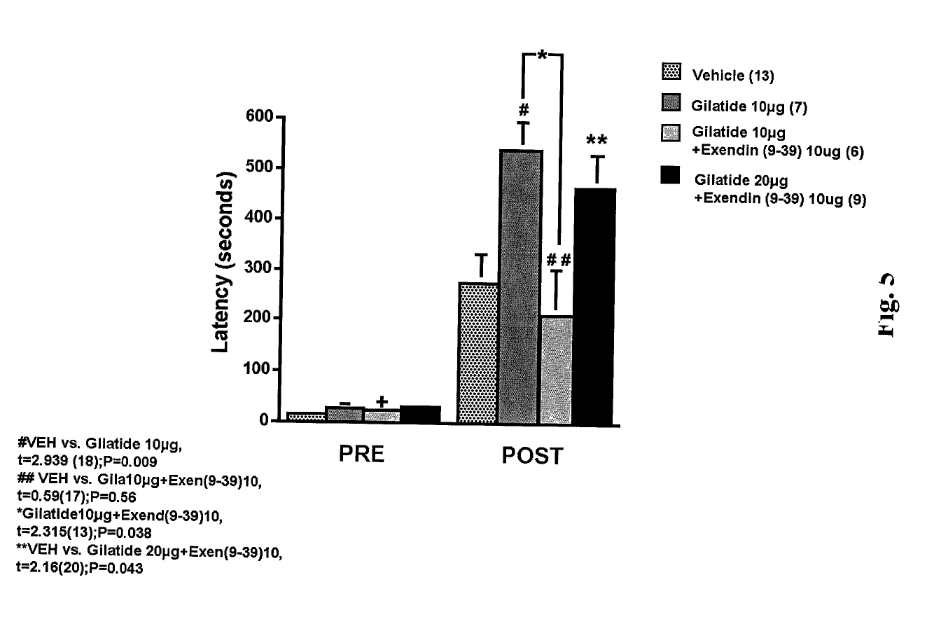

The effect of Gilatide was tested on memory consolidation by administering the peptide after shock testing. Rats (N=7-13) were preconditioned by administering a single foot shock trial (0.1 mA over 3 seconds) after entering the dark compartment of a passive avoidance apparatus. Twenty (20) minutes after the conditioning session, one group of rats was administered 10 μg/kg of Gilatide intranasally (TRN-TXT). Another group of rats (TXT-DLY-TRN) was administered this same dose of Gilatide 24 hours after the conditioning session. Both treatment groups were returned to the test apparatus 24 hours following treatment and latencies were again measured. There was no difference in latencies between the groups (p>0.05). (FIG. 4)

The effects of Gilatide when used with or without an Exendin-4 antagonist were observed and measured. Rats (N=6-13) were pretreated with either 10 μg/kg or 20 pg/kg of Gilatide with or without an Exendin-4 antagonist (10 μg/kg). A control group was administered vehicle alone. The pretreated rats were conditioned on Day 0 by administration of a single foot shock trial (0.1 mA over 3 seconds) after entry into the dark compartment of a passive avoidance apparatus (the same passive avoidance chamber used in the other experiments). The preconditioned rats were retested on 24 hours later. Co-treatment of Gilatide 10 μg/kg with an Exendin-4 antagonist (10 μg/kg) completely blocked enhancement of associative learning by Gilatide. (FIG. 5) Increasing the dose of Gilatide to 20 μg/kg surmounted the antagonism. (FIG. 5)

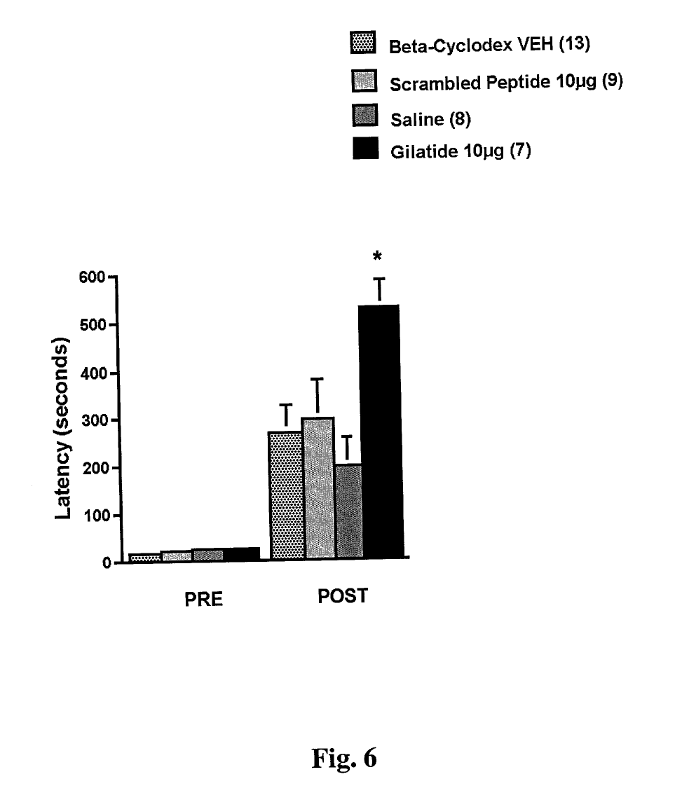

To further illustrate Gilatide's effect on passive learning in rats, rats (N=7-13) were pretreated with either Gilatide (10 μg/kg), saline (5 μl normal saline), a scrambled peptide (not matched to any active peptide) containing the same residues as Gilatide, or vehicle (5% P cyclodextrin) and conditioned on Day 0 by administration of a single foot shock trial (0.1 mA over 3 seconds) after entry into the dark compartment of a passive avoidance apparatus (the same passive avoidance chamber used in the other experiments). Twenty-four hours later the rats were returned to the apparatus and retested. The mean latencies of the groups of rats administered saline and the scrambled peptide did not differ from that of the control group (vehicle alone). (FIG. 6) In comparison, the rats administered Gilatide demonstrated a marked effect. (FIG. 6)

Locomotor Activity

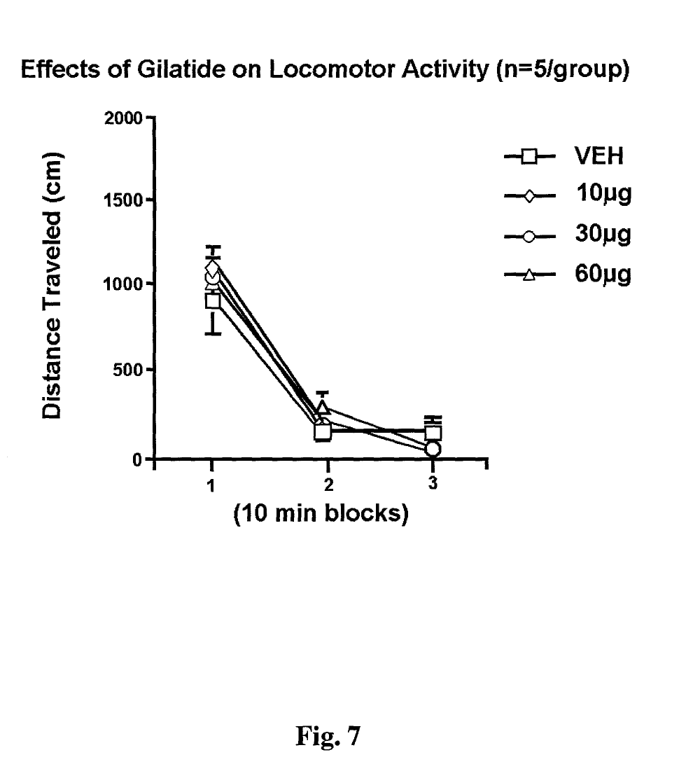

Since drugs that effect arousal and attention generally are psychomotor stimulants, Gilatide was tested in a fully automated and comprehensive locomotor activity apparatus. Rats were pretreated with either 10-60 μg/kg of Gilatide in 5% β cyclodextrin intranasally or vehicle (5% β cyclodextrin). Following pretreatment, the rats were placed for 30 minutes in an open field testing chamber (17″×17″×12″ H) where movement was detected every 50 ms by infrared photo beam emitter and detector strips at 1″ and 10″ from the bottom of the chamber. The activity chambers were linked to a PC computer and data was compiled via Activity Monitor Software (4.0, MED Associates, St. Albans, Vt). The distance traveled did not differ between treatments (p>0.05). (FIG. 7)

Pain Stimulus

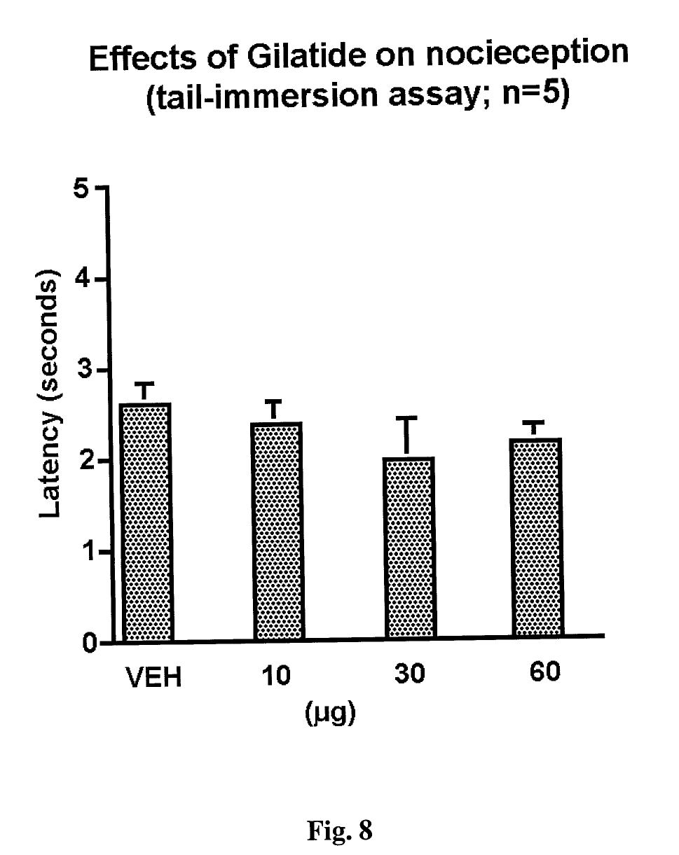

Gilatide administration was further tested in a nociceptive paradigm. Rats were pretreated with either Gilatide 10 μg/kg in 5% β cyclodextrin) intranasally or vehicle (5% β cyclodextrin). Following treatment, each rat was rolled in a towel with its tail exposed. The tail was then dipped in water maintained at 50±2° C. Latency to remove the tail from the water was measured. Latency measures did not differ between treatments. (FIG. 8)

Food and Water Intake

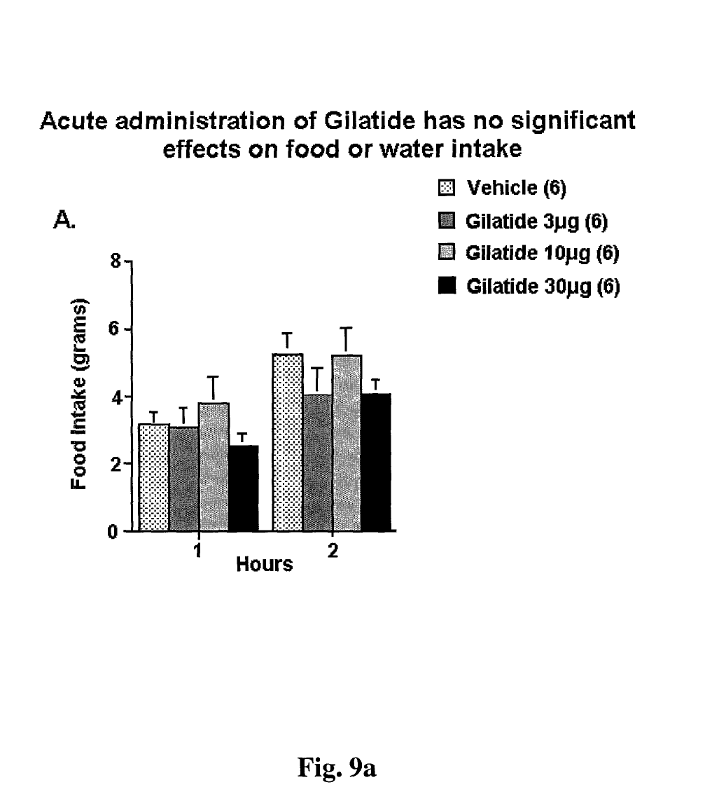

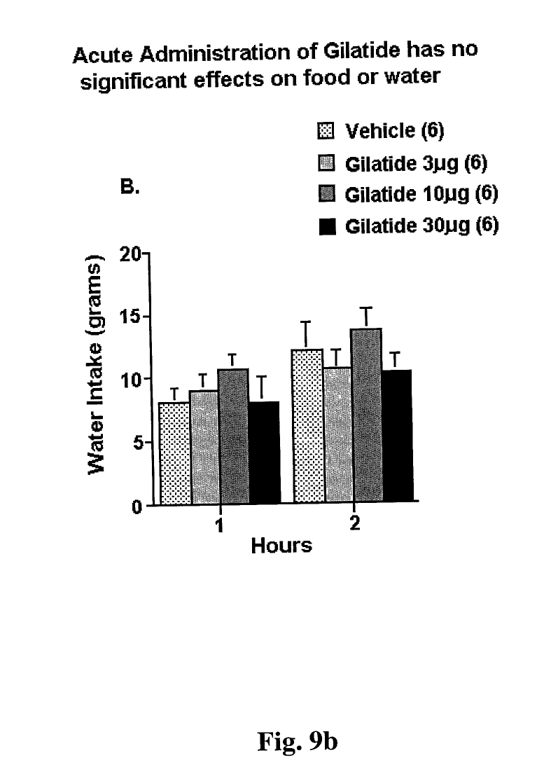

The effect of Gilatide administration was further tested by measuring the intake of food and water in rats following 18 hours of deprivation. Rats (N=6) were administered either one of three dose levels of Gilatide (3 μg/kg, 10 μg/kg, or 30 μg/kg) or vehicle and then deprived of food and water for 18 hours. Following deprivation, the rats were given access to food and water, and their intake levels of each were measured. There were no significant differences between groups treated with Gilatide compared to vehicle. (FIG. 9)

Water Maze

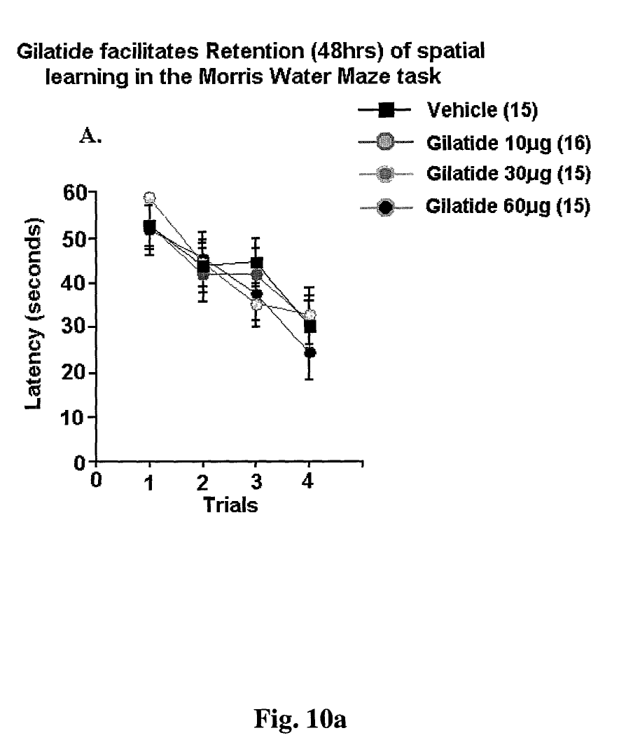

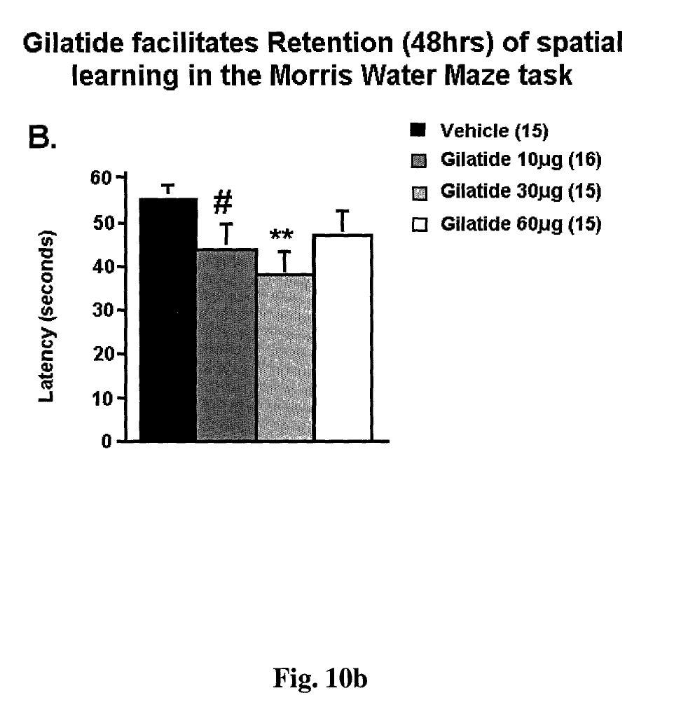

In another series of experiments, rats (N=15-16) were pretreated with either Gilatide (10 μg/kg, 30 μg/kg, or 60 μg/kg) or vehicle and then trained for fours trials in a Morris Water Maze. Two days following training, the rats were retested. Latency to find a submerged platform in the Morris Water Maze paradigm was measured. There was no difference in acquisition between groups during training. (FIG. 10) Retention tests following training yielded a trend for significance at the 10 μg/kg dose and a significant difference between Gilatide 30 μg/kg dose compared to vehicle. (FIG. 10)

CREB and MAPK Expression

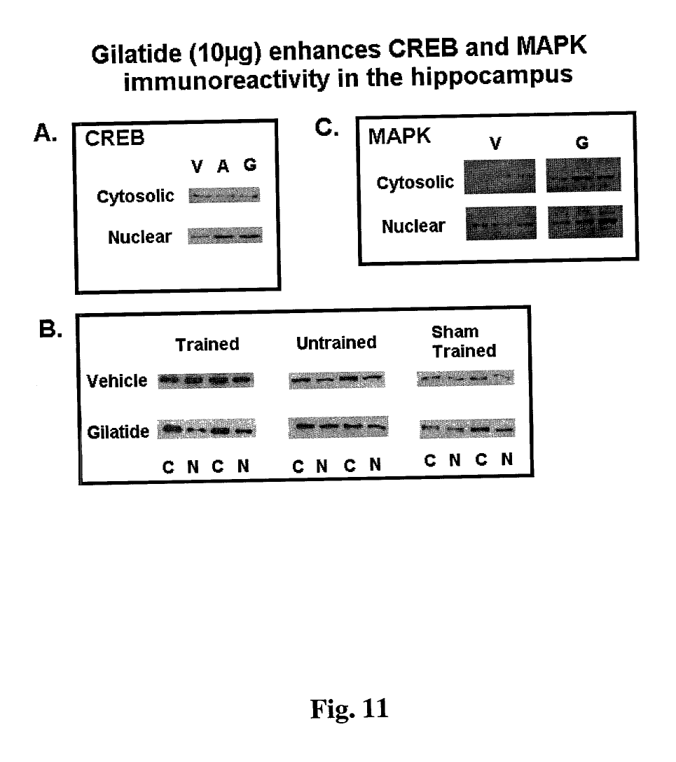

The effect of Gilatide on CREB and MAPK expression in the hippocampus was measured. In one experiment, rats were administered either vehicle, a dopamine agonist, or Gilatide 10 μg/kg intranasally. Twenty (20) minutes after treatment the rats were sacrifice and the hippocampus extracted. Samples were then separated into cytosolic and nuclear fractions and probed for CREB and MAPK protein via Western Blot Analysis. (FIG. 11 A and C) In a second experiment, rats were pretreated with either vehicle or Gilatide 10 μg/kg intranasally and then were either trained in a passive avoidance paradigm, not trained, or sham trained (shock only). The rats were sacrificed two (2) hours after training, and the hippocampus was extracted and processed. The results demonstrated that Gilatide increased CREB protein expression in hippocampal nuclear fractions 20 minutes post treatment but not at 2 hours. (FIG. 11B) Gilatide also increased MAPK protein expression in both cytosolic and nuclear fractions 20 minutes post treatment. (FIG. 11 B)

These data strongly support the use of Gilatide as a potent and long-lasting cognitive-enhancing drug. The effect of Gilatide is evident 24 hours after administration of the peptide and is still present one week after a single administration. The effect is on acquisition of memory and not consolidation. Moreover, Gilatide is devoid of behavioral activating or antinonciceptive effects and, thus, appears to be specific for memory enhancement.

Gilatide acts to increase cyclic AMP and CREB signaling in the brain. It previously has been demonstrated that drugs that facilitate CREB are neuroprotective. Thus, Gilatide, in addition to its nootropic activity (i.e., cognitive facilitation) can be neuroprotective.

Therapeutic Uses

The invention provides for treatment or prevention of various diseases, disorders, and conditions by administration of a therapeutic compound. Such therapeutics include but are not limited to: Gilatide; analogs, derivatives, fragments, and mimetics of Gilatide; and nucleic acids encoding Gilatide, and analogs, derivatives, fragments, and mimetics thereof. In an embodiment, nervous system and neurological disorders and diseases associated with neuronal loss or dysfunction are treated or prevented by administration of a therapeutic compound, specifically Gilatide or an analog, derivative, fragment, or mimetic thereof.

A polynucleotide encoding Gilatide or an analog, derivative, fragment, or mimetic thereof and its protein product can be used for therapeutic/prophylactic purposes for nervous system and neurological disorders and diseases associated with neuronal loss or dysfunction. A polynucleotide encoding Gilatide or an analog, derivative, fragment, or mimetic thereof and its protein product may be used for therapeutic/prophylactic purposes alone or in combination with other therapeutics useful in the treatment of nervous system and neurological disorders and diseases associated with neuronal loss or dysfunction.

Compounds of the instant invention are administered therapeutically (including prophylactically): (1) in diseases, disorders, or conditions involving neuronal loss or dysfunction, including, but not limited to, Parkinson's Disease, Alzheimer's Disease, Huntington's Disease, ALS, stroke, ADD, and neuropsychiatric syndromes; or (2) in diseases, disorders, or conditions wherein in vitro (or in vivo) assays indicate the utility of the peptides of the instant invention.

Therapeutic/prophylactic Methods

The invention provides methods of treatment and prophylaxis by administering to a subject an effective amount of a therapeutic, i.e., retroviral vector or peptide of the present invention. In one aspect, the therapeutic is substantially purified. The subject may be an animal, including but not limited to, animals such as cows, pigs, chickens, etc., and especially a mammal, including by not limited to, a human.

Various delivery systems are known and are used to administer a therapeutic of the invention, e.g., encapsulation in liposomes, microparticles, microcapsules, expression by recombinant cells, receptor-mediated endocytosis (see, e.g., Wu & Wu, J. Biol. Chem. 262:4429-4432, 1987), construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers.

In an embodiment where the therapeutic is a nucleic acid encoding a peptide therapeutic the nucleic acid is administered in vivo to promote expression of its encoded peptide by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide that is known to enter the nucleus (see e.g., Joliot, et al., Proc. Natl. Acad. Sci. U.S.A. 88:1864-1868, 1991), etc. (supra). Alternatively, a nucleic acid therapeutic can be introduced intracellularly and incorporated within host cell DNA for expression by homologous recombination.

The invention also provides a method of transplanting into the subject a cell genetically modified to express and secrete a peptide of the present invention. Transplantation can provide a continuous source of peptide of the instant invention and, thus, sustained treatment. For a subject suffering from neuronal loss or dysfunction, such a method has the advantage of obviating or reducing the need for repeated administration of an active peptide.

Using methods well known in the art, a cell readily can be transfected with an expression vector containing a nucleic acid encoding a peptide of the instant invention (Chang, Somatic Gene Therapy, CRC Press, Boca Raton (1995), which is incorporated herein by reference). Following transplantation into the brain, for example, the transfected cell expresses and secretes an active peptide. The cell can be any cell that can survive when transplanted and that can be modified to express and secrete Gilatide or an analog, derivative, fragment, or mimetic thereof. In practice, the cell should be immunologically compatible with the subject. For example, a particularly useful cell is a cell isolated from the subject to be treated, since such a cell is immunologically compatible with the subject.

A cell derived from a source other than the subject to be treated also can be useful if protected from immune rejection using, for example, microencapsulation or immunosuppression. Useful microencapsulation membrane materials include alginate-poly-L-lysine alginate and agarose (see, for example, Goosen, Fundamentals of Animal Cell Encapsulation and Immobilization, CRC Press, Boca Raton (1993); Tai & Sun, FASEB J. 7:1061 (1993); Liu et al., Hum. Gene Ther. 4:291 (1993); and Taniguchi et al., Transplant. Proc. 24:2977 (1992), each of which is incorporated herein by reference).

For treatment of a human subject, the cell can be a human cell, although a non-human mammalian cell also can be useful. In particular, a human fibroblast, muscle cell, glial cell, neuronal precursor cell or neuron can be transfected with an expression vector to express and secrete Gilatide or an analog, derivative, fragment, or mimetic thereof. A primary fibroblast can be obtained, for example, from a skin biopsy of the subject to be treated and maintained under standard tissue culture conditions. A primary muscle cell also can be useful for transplantation. Considerations for neural transplantation are described, for example, in Chang, supra, 1995.

A cell derived from the central nervous system can be particularly useful for transplantation to the central nervous system since the survival of such a cell is enhanced within its natural environment. A neuronal precursor cell is particularly useful in the method of the invention since a neuronal precursor cell can be grown in culture, transfected with an expression vector and introduced into an individual, where it is integrated. The isolation of neuronal precursor cells, which are capable of proliferating and differentiating into neurons and glial cells, is described in Renfranz et al., Cell 66:713-729 (1991), which is incorporated herein by reference.

Methods of transfecting cells ex vivo are well known in the art (Kriegler, Gene Transfer and Expression: A Laboratory Manual, W. H. Freeman & Co., New York (1990)). For the transfection of a cell that continues to divide such as a fibroblast, muscle cell, glial cell or neuronal precursor cell, a retroviral vector is preferred. For the transfection of an expression vector into a postmitotic cell such as a neuron, a replication-defective herpes simplex virus type 1 (HSV-1) vector is useful (During et al., Soc. Neurosci. Abstr. 17:140 (1991); Sable et al., Soc. Neurosci. Abstr. 17:570 (1991), each of which is incorporated herein by reference).

A nucleic acid encoding Gilatide or an analog, derivative, fragment, or mimetic thereof can be expressed under the control of one of a variety of promoters well known in the art, including a constitutive promoter or inducible promoter. See, for example, Chang, supra, 1995. A particularly useful constitutive promoter for high level expression is the Moloney murine leukemia virus long-terminal repeat (MLV-LTR), the cytomegalovirus immediate-early (CMV-IE) or the simian virus 40 early region (SV40).

Pharmaceutical Compositions

The pharmaceutical compositions of the invention are prepared in a manner well known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, suppositories, solutions, suspensions, powders, syrups, and the like. As used herein, the term “pharmaceutical carrier” may encompass one or more excipients. In preparing formulations of the compounds of the invention, care should be taken to ensure bioavailability of an effective amount of the agent. Suitable pharmaceutical carriers and formulation techniques are found in standard texts, such as Remington's Pharmaceutical Sciences, Mack Publishing Co., Easton, Pa.

For oral administration, the compounds can be formulated into solid or liquid preparations, with or without inert diluents or edible carrier(s), such as capsules, pills, tablets, troches, powders, solutions, suspensions or emulsions. The tablets, pills, capsules, troches and the like also may contain one or more of the following adjuvants: binders such as microcrystalline celluose, gum tragacanth or gelatin; excipients such as starch or lactose; disintegrating agents such as alsinic acid, Primogel™, corn starch and the like; lubricants such as stearic acid, magnesium stearate or Sterotex™; glidants such as colloidal silicon dioxide; sweetening agents such as sucrose or saccharin; and flavoring agents such as peppermint, methyl salicylate or fruit flavoring. When the dosage unit form is a capsule, it also may contain a liquid carrier such as polyethylene glycol or fatty oil. Materials used should be pharmaceutically pure and non-toxic in the amounts used. These preparations should contain at least 0.05% by weight of the therapeutic agent, but may be varied depending upon the particular form and may conveniently be between 0.05% to about 90% or the weight of the unit. The amount of therapeutic agent present in compositions is such that a unit dosage form suitable for administration will be obtained.

For the purpose of parenteral administration, the therapeutic agent may be incorporated into a solution or suspension. These preparations should contain at least 0.1% of the active ingredient, but may be varied to be between 0.1 and about 50% of the weight thereof. The amount of the active ingredient present in such compositions is such that a suitable dosage will be obtained.

The solutions or suspensions also may include one or more of the following adjuvants depending on the solubility and other properties of the therapeutic agent: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates; and agents for the adjustment of toxicity such as sodium chloride or dextrose. The parenteral preparation can be enclosed in ampules, disposable syringes or multiple dose vials made of glass or plastic.

The compounds can be administered in the form of a cutaneous patch, a depot injection, or implant preparation, which can be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers and synthetic silicones. Further information on suitable pharmaceutical carriers and formulation techniques are found in standard texts such as Remington's Pharmaceutical Sciences.

The exact amount of a therapeutic of the invention that will be effective in the treatment of a particular disease or disorder will depend on a number of factors and can be readily determined by the attending diagnostician, as one of ordinarily skilled in the art, by the use of conventional techniques and by observing results obtained under analogous circumstances. Factors significant in determining the dose include: the dose; the species of animal, its size, age and general health; the specific disease involved, the degree of or involvement or the severity of the disease; the response of the individual patient; the particular compound administered; the mode of administration; the bioavailability characteristics of the preparation administered; the dose regimen selected; the use of concomitant medication; and other relevant circumstances specific to the patient. Effective doses optionally may be extrapolated from dose-response curves derived from in vitro or animal model test systems. In general terms, an effective amount of a peptide of the instant invention to be administered systemically on a daily basis is about 0.1 μg/kg to about 1000 μg/kg.

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

The base peptide described herein, Gilatide, represents an example of a peptide that can be used to treat, either prophylatically or therapeutically, nervous system or neurological disorders associated with neuronal loss or dysfunction and facilitate learning, memory, and cognition. The scope of this invention is not limited to this example; the example is used to illustrate the technology of the present invention. Those skilled in the art are familiar with peptide synthesis techniques so that any analog, derivative, fragment, or mimetic that retains the biological activity of Gilatide in cellular or animal models can be used for the purposes of the present invention.

We claim:

1. A synthetic peptide, or functional analog, derivative, fragment or mimetic thereof, homologous to glucagon, Exendin- and glucagon-like peptides wherein said peptide retains bioactivity in cellular and animal models.

2. A peptide of claim 1, wherein said peptide has the sequence HSEGTFTSD (SEQ. ID. NO: 1).

3. A method of enhancing or facilitating learning, memory, and cognition in a mammal, comprising
a. administering a therapeutically effective amount of said synthetic peptide of claim 1 to said mammal; and
b. enhancing or facilitating learning, memory, and cognition in said mammal.

4. The method of claim 3, wherein administration of said therapeutically effective amount of said synthetic peptide is to a systemic site of said mammal.

5. The method of claim 4, wherein administration of said therapeutically effective amount of synthetic peptide is intranasal.

6. A method of enhancing or facilitating learning, memory, and cognition in a mammal, comprising
a. administering a therapeutically effective amount of said synthetic peptide of claim 2 to said mammal; and
b. enhancing or facilitating learning, memory, and cognition in said mammal.

7. The method of claim 6, wherein administration of said therapeutically effective amount of synthetic peptide is to a systemic site of said mammal.

8. The method of claim 7, wherein administration of said therapeutically effective amount of synthetic peptide is intranasal.

9. A method for the prophylactic and/or therapeutic treatment of a nervous system and/or neurological disease, disorder or condition associated with neuronal loss or dysfunction in a mammal, comprising
a. administering a therapeutically effective amount of said synthetic peptide of claim 1 to said mammal; and
b. treating said neuronal loss or dysfunction in said mammal.

10. The method of claim 9, wherein said nervous system and/or neurological disease, disorder, or condition is at least one of the group comprising Parkinson's Disease, Alzheimer's Disease, Huntington's Disease, ALS, stroke, ADD, and neuropsychiatric syndromes.

11. The method of claim 9, wherein administration of said therapeutically effective amount of synthetic peptide is to a systemic site of said mammal.

12. The method of claim 11, wherein administration of said therapeutically effective amount of synthetic peptide is intranasal.

13. A method for the prophylactic and/or therapeutic treatment of a nervous system and/or neurological disease, disorder or condition associated with neuronal loss or dysfunction in a mammal, comprising
a. administering a therapeutically effective amount of said synthetic peptide of claim 2 to said mammal; and
b. treating said neuronal loss or dysfunction said mammal.

14. The method of claim 13, wherein said nervous system and/or neurological disease, disorder, or condition is at least one of the group comprising Parkinson's Disease, Alzheimer's Disease, Huntington's Disease, ALS, stroke, ADD, and neuropsychiatric syndromes.

15. The method of claim 13, wherein administration of said therapeutically effective amount of synthetic peptide is to a systemic site of said mammal.

16. The method of claim 15, wherein administration of said therapeutically effective amount of synthetic peptide is intranasal.

17. A method for the prophylactic and/or therapeutic treatment of disorders, diseases, or conditions of the nervous system associated with impaired learning, memory, and cognition in a mammal, comprising
a. administering a therapeutically effective amount of said synthetic peptide of claim 1 to said mammal; and
b. facilitating cognition in said mammal.

18. The method of claim 17, wherein administration of said therapeutically effective amount of synthetic peptide is to a systemic site of said mammal.

19. The method of claim 18, wherein administration of said therapeutically effective amount of synthetic peptide is intranasal.

20. A method for the prophylactic and/or therapeutic treatment of disorders, diseases, or conditions of the nervous system associated with impaired learning, memory, and cognition in a mammal, comprising
a. administering a therapeutically effective amount of said synthetic peptide of claim 2 to said mammal; and
b. facilitating cognition in said mammal.

21. The method of claim 20, wherein administration of said therapeutically effective amount of synthetic peptide is to a systemic site of said mammal.

22. The method of claim 21, wherein administration of said therapeutically effective amount of synthetic peptide is intranasal.

23. A functional analog, derivative, fragment, or mimetic of said synthetic peptide of claim 2, wherein said functional analog, derivative, fragment, or mimetic retains the biological activity or function of SEQ. ID. NO:1 in cellular and animal models.

24. A functional analog, derivative, fragment, or mimetic of said synthetic peptide of claim 1 or claim 2, wherein said functional analog, derivative, fragment, or mimetic is modified by at least a single amino acid charge and is truncated or extended by at least one amino acid and wherein said functional analog, derivative, fragment, or mimetic retains the biological activity or function of SEQ. ID. NO:1 in cellular and animal models.

25. A functional analog, derivative, fragment, or mimetic of said synthetic peptide of claim 1 or claim 2, wherein said synthetic peptide has been modified by adding stearic acid or other residues to facilitate delivery or efficacy of said functional analog, derivative, fragment, or mimetic and wherein said functional analog, derivative, fragment, or mimetic retains the biological activity or function of SEQ. ID. NO:1 in cellular and animal models.

26. A method for the administration of the synthetic peptide of claim 1 or claim 2 to a mammal, wherein said delivery is to a systemic site of said mammal.

27. A method for the delivery of the synthetic peptide of claim 1 or claim 2 to a mammal, wherein said delivery is from an intranasal site.

28. A pharmaceutical composition comprising a pharmaceutically acceptable carrier and a therapeutically effective amount of the synthetic peptide of claim 1 or claim 2.

29. A pharmaceutical composition of claim 28, wherein said pharmaceutically acceptable carrier facilitates bioavailability and delivery of said therapeutically effective amount of the synthetic peptide to target tissues of a mammal.


SEQUENCE LISTING (1 sequence)

SEQ ID NO: 1 - protein, 9 aa, Artificial Sequence
  Synthetic peptide
HSEGTFTSD